(19) United States
(12) Patent Application Publication
(10) Pub. No.: US 2008/0085879 A1
(43) Pub. Date: Apr. 10, 2008

(54) METHODS OF TREATING ESTROGEN-RESPONSIVE CONDITIONS BY ORPHAN NUCLEAR RECEPTOR ACTIVATION

(75) Inventors: Wen Xie, Pittsburgh, PA (US); Haibiao Gong, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: University of Pittsburgh - Of the Commonwealth System of Higher Education, Pittsburgh, PA (US)

(21) Appl. No.: 11/847,163

(22) Filed: Aug. 29, 2007

Related U.S. Application Data
(60) Provisional application No. 60/841,341, filed on Aug. 31, 2006.

(51) Int. Cl. A61K 31/56; A61P 35/00

(52) U.S. Cl. 514/182

(57) ABSTRACT

The invention provides a method of treating an estrogen responsive condition comprising administration of an agonist of an orphan nuclear receptor, wherein activation of the orphan nuclear receptor inhibits estrogen activity. The invention also provides a method of reducing the size of an estrogen responsive tumor comprising administration of an agonist of an orphan nuclear receptor, wherein activation of the orphan nuclear receptor inhibits estrogen activity. The invention also provides a pharmaceutical composition comprising an agonist of an orphan nuclear receptor and a pharmaceutically acceptable vehicle, wherein activation of the orphan nuclear receptor inhibits estrogen activity.

### CROSS-REFERENCE TO RELATED APPLICATIONS

This patent application claims priority from U.S. Provisional Application for Patent No. 60/841,341, filed Aug. 31, 2006, the contents of which are incorporated herein in their entirety.

### STATEMENT REGARDING FEDERALLY SPONSORED RESEARCH AND DEVELOPMENT

This invention was made with Government support under Grant Numbers ES012479 and CA107011 awarded by the National Institutes of Health. The Government has certain rights in this invention.

### BACKGROUND OF THE INVENTION

Estrogen is an important regulator of normal physiology in women and men, and it is also known to modulate several types of cancer, as well as several non-cancer conditions. Down regulation of estrogen activity is an effective strategy for treating, and in some cases preventing, conditions exacerbated by estrogen.

In humans, the estrogen receptor (ER) is a transcription factor that activates target genes upon binding with estrogen. Estrogen-responsive cancers are characterized by the presence of estrogen receptors in the nuclei of tumor cells. ER-positive tumor cells increase in size in the presence of estrogen. Certain ER-positive non-cancerous cells also increase in size in the presence of estrogen, such as in endometriosis, leiomyomas, and gynecomastia.

Conception, contraception, and gestation rely on a delicate balance of estrogen levels as well as other hormones including progestin, gonadotropin releasing hormone (GnRH), human choronic gonadotropin (HCG), luteinizing hormone (LH), and follicle stimulating hormone (FSH). Estrogen levels that are excessively high may prevent a desired fertility outcome.

Hormone replacement therapy (HRT) regimens often contain estrogens alone or in combination with progestins. Undesirable side effects of HRT can include an increased risk of estrogen-responsive cancers.

Down regulation of estrogen activity has previously been considered as a possible means of treating such conditions, i.e., conditions associated with elevated estrogen levels.

Known methods of down regulating estrogen activity include administration of antiestrogens or aromatase inhibitors. Antiestrogens are agents that bind to the ER competitively with estrogen. Aromatase inhibitors are agents that block the production of estrogen. Side effects of antiestrogens and aromatase inhibitors can include menopause-like symptoms, and can in some cases include osteoporosis and increased risk of heart disease. Some antiestrogens have an ER-antagonist effect on some tissues with an ER-agonist effect on other tissues.

Additional methods of down regulating estrogen activity are therefore desirable.

### BRIEF SUMMARY OF THE INVENTION

The invention provides a method of treating an estrogen responsive condition comprising administration of an agonist of an orphan nuclear receptor, wherein activation of the orphan nuclear receptor inhibits estrogen activity.

The invention also provides a method of reducing the size of an estrogen responsive tumor comprising administration of an agonist of an orphan nuclear receptor, wherein activation of the orphan nuclear receptor inhibits estrogen activity.

In another embodiment, the invention provides a pharmaceutical composition comprising an agonist of an orphan nuclear receptor and a pharmaceutically acceptable vehicle, wherein activation of the orphan nuclear receptor inhibits estrogen activity.

### BRIEF DESCRIPTION OF THE FIGURES

FIG. 1—Induction of estrogen sulfotransferase (EST) by activation of Liver X Receptor (LXR). (A) Northern blot of EST mRNA of male and female wild-type (WT) and double knockout (TG) mice, further showing EST mRNA levels after treatment with LXR agonists TO1317 and 22-R hydroxycholesterol (22(R)) as compared with DMSO control; Gapdh loading controls also provided. (B) Expression of hepatic EST mRNA in wild-type (WT), LXRα/LXRβ double knockout (LXR DKO), and pregane X receptor knockout (PXR−/−) male micetreated with TO1317 (TO and DMSO (V). (C) Expression of hepatic EST mRNA in in wild-type (WT), LXRα/LXRβ double knockout (LXR DKO) female mice. (D) Expression of EST mRNA in testis of LXR DKO (TO), wild-type (WT) and wild-type treated with TO 1317 (WT+TO) mice. (E) Enzyme activity of EST in of LXR DKO (TO), wild-type (WT) and wild-type treated with TO 1317 (WT+TO) for female and male mice.

FIG. 2—Effects of LXR activation in response to estradiol (E₂) treatment (A) BrdU labeling in wild-type (WT) cells treated with DMSO (VEH). (B) BrdU labeling in wild-type (WT) cells treated with E₂. (C) BrdU labeling in wild-type (WT) cells pre-treated with TO1317 (TO) and subsequently treated with E₂. (D) BrdU labeling in VP-LXRα transgenic (TG) cells treated with DMSO (VEH). (E) BrdU labeling in VP-LXRα transgenic (TG) cells treated with E₂. (F) BrdU labeling in VP-LXRα transgenic (TG) cells pre-treated with TO1317 (TO) and subsequently treated with E₂. (G) mRNA expression of receptors in response to E₂ treatment. (H) Photograph of uterine enlargement in E₂ treated wild-type (WT) mice as compared to VP-LXRα treated transgenic (TG) mice. (I) Percentage by bodyweight of uterine enlargement in E₂ treated wild-type (WT) mice as compared to VP-LXRα treated transgenic (TG) mice.

FIG. 3—EST is a transcriptional target of LXRα (A) Luciferase reporter genes that contain the mEst gene promoter (ESTP) sequences were transfected into HepG2 cells together with the LXRα expression vector. Fold inductions of TO1317 over solvent control are labeled. (B) The partial DNA sequence of the ESTP. The DR-4 element is capitalized and the mutant variant is shown with the mutated nucleotides underlined. The LXRE from the Srebp-1c promoter is also shown. (C) The LXRα/RXRα heterodimers bind to Est/DR-4 as revealed by EMSA. (D) Recruitment of mLXRα onto the mEst promoter as revealed by CHIP analysis. HA-tagged LXRα or the HA vector control was used to transfect wild type mouse livers by a hydrodynamic gene delivery method. Mice were treated with DMSO or TO1317 for 9.5 h before sacrificing. CHIP was performed with the use of an anti-HA antibody. Lanes represent individual mice. (E) LXRα activates the tk-Est/DR4 promoter in HepG2 cells and the 2. 1-kb promoter in the mouse livers. Cell transfection results shown are fold induction over solvent and represent the averages and standard deviation from triplicate assays. n=6 per group for the promoter liver transfection.

FIG. 4—LXR agonist TO1317 inhibited MCF-7/VEGF cell tumorigenicity in nude mice. (A) Growth kinetics of the MCF-7/VEGF tumors in the presence of various hormone and drug treatments. Tumor volumes were measured at the indicated times. Results are presented as mean±SD. Each group contains at least 9 mice. **P<0.005, compared to the TO1317 treatment group. (B) Appearance of representative E₂-induced tumors in the vehicle- or TO1317-treated mice. (C) Representative BrdU immunostaining of E2-induced tumors in the vehicle- or TO1317-treated mice. (D) Quantitation of BrdU-positive nuclei in the E₂-induced tumors 25 d post inoculation. (E) Serum levels of E₂ from mice in FIG. 4a at the time of sacrificing (35 d post inoculation).

FIG. 5—Induction of EST by activation of glucocorticoid receptor (GR). Northern blot of EST mRNA level as increased by dexamethasone (DEX) treatment in livers of both male and female mice. The expression increase is independent of pregane X receptor (PXR) as DEX still induced EST expression in PXR−/− mice. CYP3A is a PXR target gene that was induced by both PCN and DEX. Gapdh loading controls also provided.

FIG. 6—Induction of EST by administration of GR agonist in wild type (WT) but not in GR knockout (GR−/−) mice. Induction is seen in liver (liv) but not in testis (tes). Induction of PXR target gene CYP3A by DEX still exists in GR−/− mice. Gapdh loading controls also provided.

FIG. 7—EST enzymatic activity increased by dexamethasone (DEX) treatment in livers of male and female mice.

FIG. 8—Decreased tumorigenicity in MCF-7/VEGF xenografted cells treated with DEX.

FIG. 9—Serum estradiol level in mice during tumorigenesis, in control and dexamethasone treated mice.

FIG. 10—GR agonist dexamethasone (DEX) regulates EST in human cells. (A) RT-PCR of EST mRNA harvested from primary human hepatocytes treated with DMSO (control) and DEX. (B) RT-PCR of EST mRNA harvested from human MCF7 breast cancer cells that have been transfected with empty vector CMX, GR, or activated VPGR.

FIG. 11—EST mRNA expression in the liver of female CD1 mouse.

FIG. 12—EST mRNA expression in the liver and testis of male CD-1 mouse.

### DETAILED DESCRIPTION OF THE INVENTION

The invention provides a method of treating an estrogen responsive condition comprising administration of an agonist of an orphan nuclear receptor, wherein activation of the orphan nuclear receptor inhibits estrogen activity.

The estrogen responsive condition can be any condition that is exacerbated by increased estrogen levels, or any condition that is improved by reducing estrogen levels. Preferably, the estrogen responsive condition is breast cancer, lung cancer, uterine cancer, and prostate cancer. Most preferably, the estrogen responsive condition is breast cancer.

Other contemplated estrogen responsive conditions include but are not limited to leiomyoma and endometriosis, adverse side effects of hormone replacement therapy, and fertility or infertility. One skilled in the art can identify appropriate conditions for treatment based on the presence or absence of estrogen receptors in tumor cells, wherein the presence of estrogen receptors in tumor cells indicates that the tumor cells are estrogen responsive. In other cases, one skilled in the art may determine whether a condition can be treated effectively with the therapies of the present invention based on the patient's hormone levels or symptoms.

The contemplated methods of treating estrogen responsive conditions can be used to reduce the size of estrogen responsive cells and tissues. Preferably, the methods are used to reduce the size of estrogen responsive tumors.

Generally, an orphan nuclear receptor is a nuclear receptor whose ligand is unidentified or unnecessary. In the present invention, the orphan nuclear receptor can be selected from the group consisting of Liver X Receptors (LXR) and glucocorticoid receptors (GR). LXR and GR are believed to regulate estrogen sulfotransferase (EST) and control estrogen homeostasis, an endocrine role distinct from the known function of LXRs in cholesterol and lipid homeostasis and inflammation. Tontonoz, P. et al., Mol. Endocrinol. 17:985-993 (2003); Tong, M. H. et al., Nat. Med. 11:153-159 (2005); Falany, J. L., et al., Breast Cancer Res. Treat. 74:167-176 (2002); Qian, Y., et al., J. Pharmacol. Exp. Ther. 286:555-560 (1998); Falany, J. L., et al., Oncol. Res. 9:589-596 (1997). Without being bound by any particular theory, EST-mediated sulfation is thought to be a metabolic pathway to deactivate estrogens.

The Liver X Receptor can be selected from the group consisting of LXRα (also referred to as LXRa) and LXRβ (also referred to as LXRb).

The agonist can be any agonist of a Liver X Receptor. Exemplary LXR agonists include GW3965 (Collins, J. L. et al., J. Med. Chem., 45:1963-1966 (2002)), TO1317 (T0901317) (Repa, J. J. et al. Science 289:1524-1529 (2000)), 22(R)-hydroxycholesterol, 25-hydroxycholesterol, or another LXR agonist. The LXR agonist can be specific or non-specific. Specific LXR agonists can be agonists of LXRα, LXRβ, or both. Non-specific agonists are also contemplated, including certain LXR agonists that are also agonists of PXR (Pregane X Receptor).

The agonist can also be any agonist of a glucocorticoid receptor. Exemplary GR agonists include dexamethasone, ZK216348 (see Schacke, et al., Proc. Nat. Acad. Sci. 101:227-232 (2004)), RU28362 (see Roozendaal, et al., Proc. Nat. Acad. Sci. 96:11642-11647 (1999)), cortisol, prednisone, prednisolone, or another GR agonist.

Several types of combination treatments are contemplated in the methods of the present invention. It will be understood that any combination of the methods described herein can be used to treat any estrogen-responsive condition, such as those listed above. More particularly, any combination of the methods described herein may be employed to reduce the size of estrogen responsive cells or tissues, such as estrogen responsive tumors.

In some embodiments, the invention can provide a method of treating an estrogen responsive condition comprising administration of an agonist of an orphan nuclear receptor, and administration of a second agonist of an orphan nuclear receptor, wherein the first agonist is different from the second agonist. The first agonist and the second agonist can be administered as part of a treatment regimen for any estrogen responsive condition, such as those listed above.

In some embodiments, the first orphan nuclear receptor and the second orphan nuclear receptor are different (i.e., “first” and “second” orphan nuclear receptors). Preferably, the first orphan nuclear receptor is LXR and the second orphan nuclear receptor is GR. In other embodiments, both orphan nuclear receptors are LXR. In still other embodiments, both orphan nuclear receptors are GR.

The first agonist and the second agonist can be any agonist of the corresponding orphan nuclear receptor as described above. In one embodiment, the first orphan nuclear receptor is LXR and the second orphan nuclear receptor is GR, and the first and second agonists are agonists of LXR and GR, respectively. In a preferred embodiment, the first agonist is GW3965 and the second agonist is dexamethasone. In another embodiment, the first agonist and the second agonist are different agents that act on the same receptor. The first agonist and the second agonist can be administered simultaneously or consecutively.

The inventive method for treating estrogen responsive conditions can be combined with administration of chemotherapy. The chemotherapy can be any suitable type of drug therapy known to one skilled in the art. Preferably, the chemotherapy is selected from the group consisting of an antiestrogen, an aromatase inhibitor, and a cytotoxic chemotherapy.

In some embodiments, the chemotherapy is an antiestrogen. The antiestrogen can be any pharmaceutically acceptable antiestrogen, including derivatives and salts of known antiestrogens. The antiestrogen can be tamoxifen, toremifene, raloxifene, droloxifene, idoxifine, nafoxidine, levomeloxifene, clomiphene, CI-680, CI-628, CN-55,956-27, MER-25, U-11,555A, U-11,100A, ICI-46,669, ICI-46,474, diphenolhydrochrysene, erythro-MEA, Parke Davis CN-35,945, allenolic acid, cyclofenil, ethamoxytriphetol, triparanol, and the like.

In other embodiments, the chemotherapy is an aromatase inhibitor. The aromatase inhibitor can be any pharmaceutically acceptable aromatase inhibitor, including derivatives and salts of known aromatase inhibitors. The aromatase inhibitor can letrozole, anastrozole, exemestane, raloxifene, fadrozole, lentaron, formestane, rivizor, vorozole, fulvestrant, and the like.

In still other embodiments, the chemotherapy is a cytotoxic chemotherapy. Classes of compounds that can be used as cytotoxic agents include, but are not limited to the following:

Alkylating agents useful as cytotoxic chemotherapy can be, without limitation, nitrogen mustards, ethylenimine derivatives, alkyl sulfonates, nitrosoureas and triazenes), including Uracil mustard, Chlormethine, Cyclophosphamide (Cytoxan.RTM.), Ifosfamide, Melphalan, Chlorambucil, Pipobroman, Triethylene-melamine, Triethylenethiophosphoramine, Busulfan, Carmustine, Lomustine, Streptozocin, Dacarbazine, Temozolomide, and the like.

Antimetabolites useful as cytotoxic chemotherapy can be, without limitation, folic acid antagonists, pyrimidine analogs, purine analogs and adenosine deaminase inhibitors, including Methotrexate, 5-Fluorouracil, Floxuridine, Cytarabine, 6-Mercaptopurine, 6-Thioguanine, Fludarabine phosphate, Pentostatine, Gemcitabine, and the like.

Natural products and their derivatives useful as cytotoxic chemotherapy can be, without limitation, vinca alkaloids, antitumor antibiotics, enzymes, lymphokines and epipodophyllotoxins, including Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol.RTM.), Mithramycin, Deoxyco-formycin, Mitomycin-C, L-Asparaginase, Interferons (especially IFN-a), Etoposide, Teniposide, and the like.

Other anti-proliferative cytotoxic agents can include navelbene, CPT-11, anastrazole, letrazole, capecitabine, reloxafine, cyclophosphamide, ifosamide, and droloxafine.

Microtubule affecting agents interfere with cellular mitosis and are well known in the art for their anti-proliferative cytotoxic activity, and as such can be used in the inventive methods. Microtubule affecting agents useful in the invention include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol.RTM., NSC 125973), Taxol.RTM. derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, and discodermolide (see Service, (1996) Science, 274:2009) estramustine, nocodazole, MAP4, and the like. Examples of such agents are also described in the scientific and patent literature, see, e.g., Bulinsli (1997) J. Cell Sci. 110:3055 3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem 271:29807-29812.

Cytotoxic agents suitable for use in the methods and compositions of this invention include, but are not limited to, microtubule-stabilizing agents such as paclitaxel (also known as Taxol.RTM.), docetaxel (also known as Taxotere.RTM.), 7-O-methylthiomethylpaclitaxel (disclosed in U.S. Pat. No. 5,646,176), 4-desacetyl-4-methylcarbonatepaclitaxel, C-4 methyl carbonate paclitaxel (disclosed in WO 94/14787), epothilone A, epothilone B, epothilone C, epothilone D, desoxyepothilone A, desoxyepothilone B, and derivatives thereof; and microtubule-disruptor agents.

Additional cytotoxic agents that can be used in the inventive method include melphalan, hexamethyl melamine, thiotepa, cytarabin, idatrexate, trimetrexate, dacarbazine, L-asparaginase, camptothecin, topotecan, bicalutamide, flutamide, leuprolide, pyridobenzoindole derivatives, interferons, and interleukins. Preferred classes of antiproliferative cytotoxic agents are the EGFR inhibitors, Her-2 inhibitors, CDK inhibitors, and Herceptin.RTM. (trastuzumab). Some preferred anti-proliferative cytostatic agents are paclitaxel, cis-platin, carboplatin, epothilones, gemcytabine, CPT-11,5-fluorouracil, tegafur, leucovorin, and EGFR inhibitors such as Iressa.RTM. (ZD 1839, 4-(3-chloro-4-fluorophenylamino)-7-methoxy-6-(3-(4-morpholinyl)propoxy)quinazoline and OSI-774 (4-(3-ethynylphenylamino)-6,7-bis(2-methoxyethoxy)quinazoline).

Most preferably, the cytotoxic chemotherapy is selected from the group consisting of methotrexate, cyclophosphamide, 5-flurouracil, doxorubicin, paclitaxel, and docetaxel.

Additionally, the inventive method for treating estrogen responsive conditions can be combined with surgical treatment or radiation therapy. Under these circumstances, the method of treatment can be adjuvant to surgery or radiation. Treatment can begin after surgery and/or radiation has been completed. In other cases, treatment can begin before surgery and/or radiation has begun. In a preferred embodiment, treatment of patients suffering from tumors is begun prior to surgery so that the tumor will be reduced in size, decreasing the need for radical surgery.

Agonists of orphan nuclear receptors (“agonists”) can be administered in any suitable dosage as determined by the treating physician. The correct dosage will depend on several factors including but not limited to the strength of the agonist(s) selected, the amount and type of other agents to be administered to the patient, the patient's estradiol levels, and the dosage regimen planned. LXR agonists generally are administered in dosages less than about 40 mg/kg to minimize agonism of other receptors (e.g., PXR). More typically, in the context of the present invention, LXR agonists are administered at dosages not exceeding about 12 mg/kg, such as not exceeding about 10 mg/kg. However, suitable dosages of LXR agonists for use in the context of the present invention typically are not less than about 1 mg/kg, such as minimally about 2 mg/kg. GR agonists, such as dexamethasone, can be administered at dosages of at least about 5 mg/kg, more typically at least about 10 mg/kg and maximally about 30 mg/kg, such as maximally about 20 mg/kg. Suitable dosages of GR agonists typically fall into a range of about 10 mg/kg to about 15 mg/kg. However, it will be understood by one skilled in the art that the dosage will be highly tailored to the circumstances of the patient.

In all embodiments of the present invention, a variety of administration schedules are possible, including embodiments where administration of an agonist of an orphan nuclear receptor is combined with an additional treatment component, such as a second agonist, chemotherapy, surgery or radiation. An agonist can be administered as a single dose treatment or as part of a daily, weekly, bi-weekly, or monthly treatment regimen. An agonist can be administered for consecutive days, such as 2, 3, 4, 5, 6, 7, 8, 9, 10, 11, 12, 13, 14, or more than 14 consecutive days. An agonist can be administered in therapy cycles including one or more days, weeks, or months of treatment followed by one or more days, weeks, or months of rest period. In some embodiments, the treatment schedule is determined by the patient's estradiol levels, wherein an agonist is administered upon detection of a hormone level greater than desired.

In embodiments where treatment with a first agonist is combined with treatment with a second agonist, the two agonists can be administered simultaneously on any schedule as described above, or in sequence. Where more than one agonist is administered in sequence, the agonists can be administered on alternating days, weeks, or months, in the schedule described above. They can also be administered in alternating therapy cycles.

In embodiments where treatment with an agonist is combined with chemotherapy, the agonist can be administered prior to starting chemotherapy, during chemotherapy, or after chemotherapy. If the agonist is administered during chemotherapy, it can be administered in conjunction with the chemotherapy. In some embodiments, agonist therapy cycles can be alternated with chemotherapy cycles.

Likewise, in embodiments where treatment with an agonist is combined with radiation therapy, the agonist can be administered prior to starting radiation therapy, during the course of radiation therapy, or after radiation therapy.

For delivery to patients, an agonist of an orphan nuclear receptor is preferably formulated in a pharmaceutically acceptable formulation. Accordingly, the invention also provides a pharmaceutical composition comprising an agonist of a nuclear receptor and a pharmaceutically acceptable vehicle, wherein the nuclear receptor is an inhibitor of estrogen activity. The nuclear receptor can be selected from the group consisting of Liver X Receptors (LXR) and glucocorticoid receptors (GR). The Liver X Receptor is selected from the group consisting of LXRα and LXRβ. The agonist can be any agonist described above. Compositions comprising multiple agonists are also provided, as described above.

The pharmaceutical compositions of the present invention can be in any form that allows for the composition to be administered to a patient safely and efficaciously. Pharmaceutical composition of the invention are preferably formulated so as to allow the active ingredients contained therein to be bioavailable upon administration of the composition to a patient. For example, the composition can be in the form of a solid, liquid or gas (aerosol). Typical routes of administration include, without limitation, oral, topical, parenteral, and transmucosal. Parenteral administration can include subcutaneous injections, intravenous, intramuscular, epidural, intrasternal injection or infusion techniques. Transmucosal administration can include sublingual, intranasal, rectal, vaginal, and pulmonary administration. Compositions that will be administered to a patient take the form of one or more dosage units, where for example, a tablet, capsule or cachet can be a single dosage unit, and a container in aerosol form can hold a plurality of dosage units.

Materials used in preparing the pharmaceutical compositions should be pharmaceutically pure and non-toxic in the amounts used. The inventive compositions can include one or more active agents in addition to the agonist(s). For instance, a cytotoxic chemotherapeutic can be combined with an LXR or GR agonist of the invention, to provide a composition useful in reducing tumor size prior to surgery. It will be evident to those of ordinary skill in the art that the optimal dosage of the active ingredient(s) in the pharmaceutical composition will depend on a variety of factors. Relevant factors include, without limitation, the type of subject (e.g., human), the particular form of the active ingredient, the manner of administration and the composition employed.

In general, the pharmaceutical composition includes an agonist of an orphan nuclear receptor as described herein, in admixture with one or more carriers. The carrier(s) can be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, e.g., inhalatory administration.

When formulated for oral administration, the composition is preferably in either solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, cachet, chewing gum, wafer, lozenges, or the like form. Such a solid composition will typically contain one or more inert diluents or edible carriers. In addition, one or more of the following components can be present: binders such as syrups, acacia, sorbitol, polyvinylpyrrolidone, carboxymethylcellulose, ethyl cellulose, microcrystalline cellulose, gum tragacanth or gelatin, and mixtures thereof; excipients such as starch, lactose or dextrins, disintegrating agents such as alginic acid, sodium alginate, Primogel, corn starch and the like; lubricants such as magnesium stearate or Sterotex; fillers such as lactose, mannitols, starch, calcium phosphate, sorbitol, methylcellulose, and mixtures thereof; lubricants such as magnesium stearate, high molecular weight polymers such as polyethylene glycol, high molecular weight fatty acids such as stearic acid, silica, wetting agents such as sodium lauryl sulfate, glidants such as colloidal silicon dioxide; sweetening agents such as sucrose or saccharin, a flavoring agent such as peppermint, methyl salicylate or orange flavoring, and a coloring agent.

When the composition is in the form of a capsule, e.g., a gelatin capsule, it can contain, in addition to materials of the above type, a liquid carrier such as polyethylene glycol or a fatty oil.

The composition can be in the form of a liquid, e.g., an elixir, syrup, solution, aqueous or oily emulsion or suspension, or even dry powders which can be reconstituted with water and/or other liquid media prior to use. The liquid can be for oral administration or for delivery by injection, as two examples. When formulated for oral administration, preferred compositions contain, in addition to the present compounds, one or more of a sweetening agent, thickening agent, preservative (e.g., alcyl p-hydoxybenzoate), dye/colorant and flavor enhancer (flavorant). In a composition formulated to be administered by injection, one or more of a surfactant, preservative (e.g., alcyl p-hydroxybenzoate), wetting agent, dispersing agent, suspending agent (e.g., sorbitol, glucose, or other sugar syrups), buffer, stabilizer and isotonic agent can be included. The emulsifying agent can be lecithin or sorbitol monooleate, or any other known emulsifying agent.

The liquid pharmaceutical compositions of the invention, whether they be solutions, suspensions or other like form, can include one or more of the following components: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred diluent. An injectable pharmaceutical composition is preferably sterile.

The pharmaceutical composition can be administered topically, in which case the carrier can comprise a solution, emulsion, ointment, cream or gel base. The base, for example, can comprise one or more of the following: petrolatum, lanolin, polyethylene glycols, beeswax, mineral oil, diluents such as water and alcohol, and emulsifiers and stabilizers. Thickening agents can be present in a pharmaceutical composition for topical administration. If formulated for transdermal administration, the composition can include a transdermal patch or iontophoresis device.

The composition can be administered rectally or vaginally in the form, e.g., of a suppository which will melt in the rectum or vagina and release the drug. The composition for suppository administration can contain an oleaginous base as a suitable nonirritating excipient. Such bases include, without limitation, lanolin, cocoa butter and polyethylene glycol. Low-melting waxes are preferred for the preparation of a suppository, where mixtures of fatty acid glycerides and/or cocoa butter are suitable waxes. The waxes can be melted, and the agonist is dispersed homogeneously therein by stirring. The molten homogeneous mixture is then poured into convenient sized molds, allowed to cool and solidify.

The composition can include various materials which modify the physical form of a solid or liquid dosage unit. For example, the composition can include materials that form a coating shell around the active ingredients. The materials which form the coating shell are typically inert, and can be selected from, for example, sugar, shellac, and other enteric coating agents. Alternatively, the active ingredients can be encased in a gelatin capsule or cachet.

The pharmaceutical composition of the present invention can consist of gaseous dosage units, e.g., it can be in the form of an aerosol. Aerosol formulations can include a variety of systems ranging from those of colloidal nature to systems consisting of pressurized packages. Delivery can be by a liquefied or compressed gas or by a suitable pump system which dispenses the active ingredients. Aerosols of compounds of the invention can be delivered in single phase, bi-phasic, or tri-phasic systems in order to deliver the active ingredient(s). Delivery of the aerosol includes the necessary container, activators, valves, subcontainers, and the like, which together can form a kit. Preferred aerosols can be determined by one skilled in the art, without undue experimentation.

The pharmaceutical compositions can be prepared by methodology well known in the pharmaceutical art. The agonists of the invention can be in the form of a solvate in a pharmaceutically acceptable solvent such as water or physiological saline. Alternatively, the compounds can be in the form of the free base or in the form of a pharmaceutically acceptable salt such as the hydrochloride, sulfate, phosphate, citrate, fumarate, methanesulfonate, acetate, tartrate, maleate, lactate, mandelate, salicylate, succinate and other salts known in the art. The appropriate salt would be chosen to enhance bioavailability or stability of the compound for the appropriate mode of employment (e.g., oral or parenteral routes of administration).

A composition formulated to be administered by injection can be prepared by combining the agonist with water, and preferably buffering agents, so as to form a solution. The water is preferably sterile pyrogen-free water. A surfactant can be added to facilitate the formation of a homogeneous solution or suspension. Surfactants are compounds that facilitate dissolution or homogeneous suspension of the agonist in the aqueous delivery system. Other carriers for injection include, without limitation, sterile peroxide-free ethyl oleate, dehydrated alcohols, propylene glycol, as well as mixtures thereof.

Suitable pharmaceutical adjuvants for the injecting solutions include stabilizing agents, solubilizing agents, buffers, and viscosity regulators. Examples of these adjuvants include ethanol, ethylenediaminetetraacetic acid (EDTA), tartrate buffers, citrate buffers, and high molecular weight polyethylene oxide viscosity regulators. These pharmaceutical formulations can be injected intramuscularly, epidurally, intraperitoneally, or intravenously.

The following examples further illustrate the invention but, of course, should not be construed as in any way limiting its scope.

### EXAMPLE 1

This example demonstrates that estrogen sulfotransferase (EST) is a transcriptional target of LXR, and that EST can be increased upon activation of LXR by administration of LXR agonists.

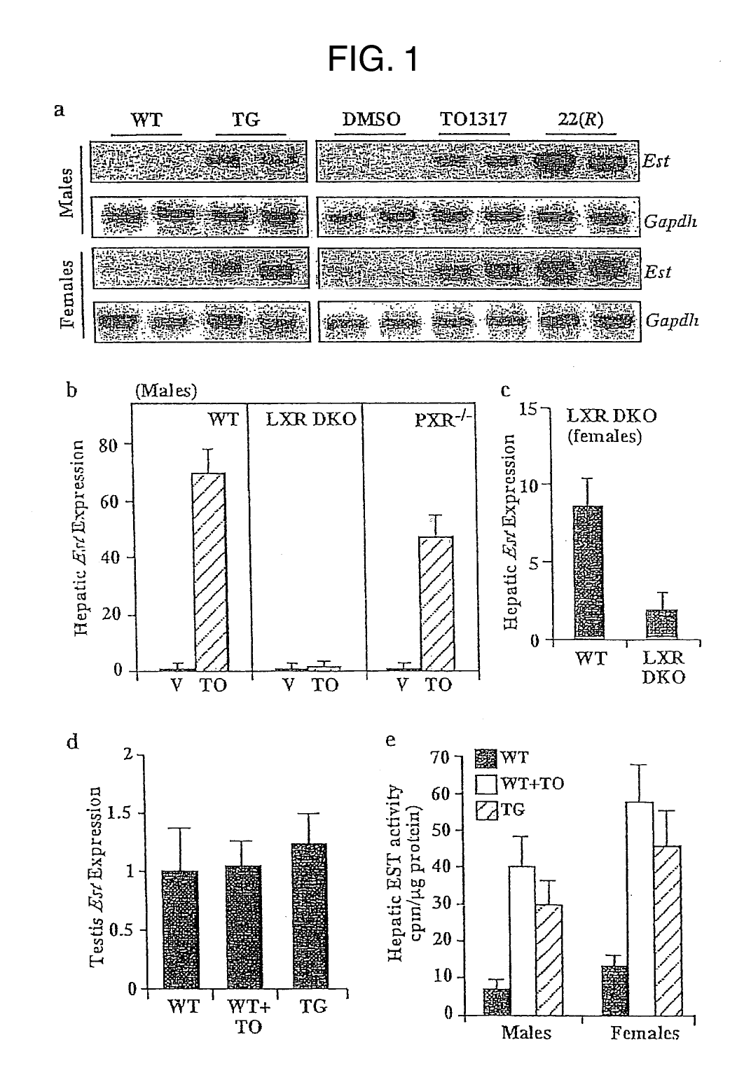

Transgenic mice, having a mixed background of C57BL/6J and 129/SvImJ, were created expressing activated LXRa in the liver under the control of the fatty acid binding protein (FABP) promoter. (FIG. 1) EST levels in liver cells of the transgenic mice were compared to wild-type mice using Northern blot analysis and real-time PCR, as described in Gong, H. et al. Mol. Endocrinol. 20:279-290 (2006) and Zhou, J. et al. J Biol Chem. 281: 15013-15020 (2006), respectively. In the transgenic mice expressing activated LXRα, the liver expression of EST was markedly up-regulated as compared to wild-type mice.

Wild-type mice were treated with LXR agonists TO1317 (T0901317) or 22(R)-hydroxycholesterol (FIG. 1a), and EST levels were compared to DMSO-treated control mice as described above. As in the transgenic mice, EST was up-regulated compared to control mice.

LXRα and LXRβ double knockout (LXR DKO) mice were created as described in Peet, D. J. et al., Cell 93:693-704 (1998). When the LXR DKO mice and wild-type mice were treated with TO1317, the double-knockout mice showed no increase in EST levels as compared with the control mice. (FIG. 1b) The LXR DKO females also showed decreased basal expression of EST (FIG. 1c), suggesting that LXR is required for both the basal and inducible EST expression. These effects were not observed in the testis of male mice, however. (FIG. 1d)

The effects of genetic and pharmacological activation of LXR on EST expression were also confirmed by a sulfotransferase enzymatic assay using estrone as the substrate (FIG. 1e). The sulfotransferase assay was carried out using [³⁵S]pAPS (Perkin Elmer) as previously described. Saini, S. P. et al., Mol. Pharmacol. 65:292-300 (2004). In brief, 20 μg/ml of total liver cytosolic extract was used with 1 μM of estrone substrate. After the reactions, free [³⁵S]-PAPS was removed by extracting with ethyl acetate. The aqueous phase was then counted in scintillation counter for radioactivity. The LXR agonists induced EST expression and activity as efficiently as the transgene (FIGS. 1a and 1e).

### EXAMPLE 2

This example demonstrates the effect of LXR-mediated EST activation on estrogen deprivation.

Ovariectomized wild type and VP-LXRα transgenic females were subjected to uterine estrogen response measurements that include epithelial proliferation by BrdU labeling and estrogen responsive gene expression by real-time PCR. Five-week old virgin females were subjected to ovariectomies. Seven days after the surgery, mice were given a single s.c. injection of 17β-estradiol (E₂) (25 μg/mouse). 18 h after the E₂ injection, mice were given a single i.p. injection of bromodeoxyuridine (BrdU) (60 mg/kg) and sacrificed 2 h after. One uterine horn was harvested for histology and measurement of cell proliferation by BrdU immunostaining (Xie, W., et al., Mol. Endocrinol. 11: 1766-1781 (1997)) and the other was harvested for RNA extraction and gene expression analysis by real-time PCR. In the uterotropic bioassay, 3-week-old virgin female mice received daily s.c. injections of vehicle or E₂ (5 μg/kg) for 3 d. Mice were then sacrificed 24 h after the last E₂ dose and the uteri were dissected, weighed, and photographed. When necessary, mice were subjected to daily treatment of TO1317 (50 mg/kg, i.p. injection) starting 3 d prior to the E2 treatment and continued until the completion of the experiments.

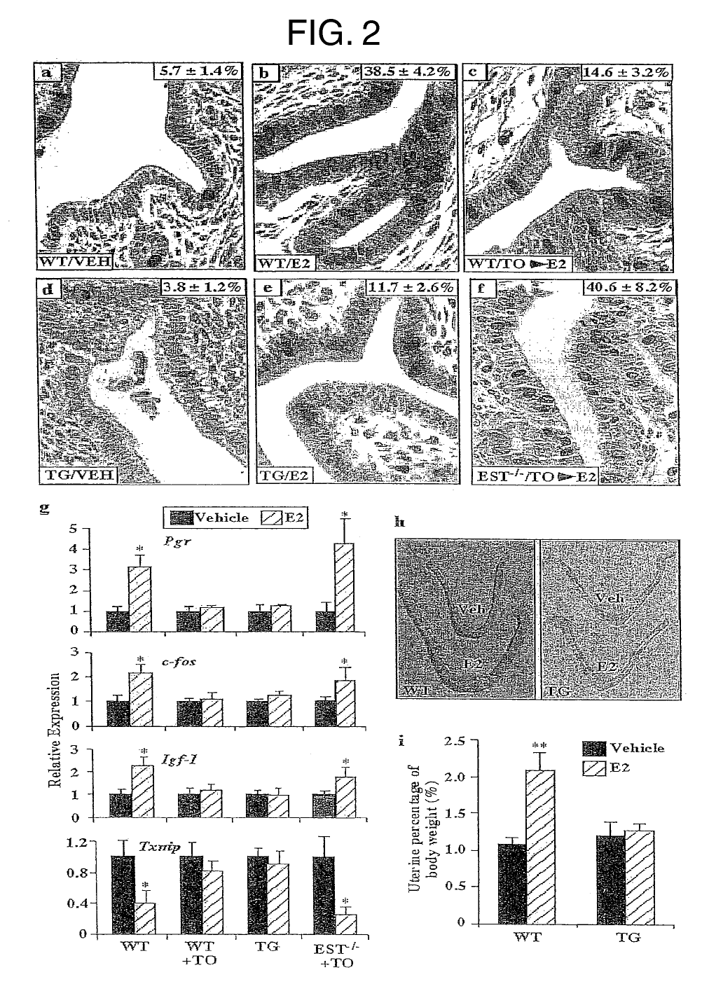

As shown in FIG. 2, E₂ treatment in wild type mice increased BrdU labeling as expected (FIG. 2b). However, the E₂ effect was compromised in both the TO1317 pre-treated wild type (FIG. 2c) and mock-treated VP-LXRα transgenic (FIG. 2e) mice. The transgene or TO1317 treatment alone, in the absence of E₂, had little effect on the basal proliferation (FIG. 2d, and data not shown). When the TO1317 treatment was performed in the EST null mice (Tong, et al., Endocrinology 145:2487-2497 (2004)) or when the VP-LXRα transgene was bred into the EST null background, the TO1317 and transgenic effects on the E₂ response were abolished (FIG. 2f, and data not shown), suggesting that EST expression is required for the LXR effect. Activation of LXRs also led to an inhibition of estrogen-responsive uterine gene expression. The mRNA expression of progesterone receptor (Pgr), c-fos, and insulin-like growth factor 1 (Igf-1) was induced and Txnip was suppressed by E₂ in ovariectomized wild type female mice as expected (FIG. 2g). Klotz, D. M. et al., J. Biol. Chem. 277:8531-8537 (2002); Deroo, B. J., et. al., Endocrinology 145:5485-5492 (2004); Kirkland, et al., Mol. Pharmacol. 43:709-714 (1993); Mendoza-Rodriguez, et al., Contraception 59, 339-343 (1999). Consistent with the cell proliferation results, the E₂ effects on the expression of these E₂-responsive genes were abolished in the TO1317-treated wild type and mock-treated VP-LXRα transgenic mice, and the TO1317 effect was abolished in the EST null mice (FIG. 2g). In another estrogen-dependent uterotropic bioassay (see Korach, K. S., Science 266:1524-1527 (1994)), estrogen treatment caused typical uterine enlargement (water imbibition) in the wild type female mice but this uterotropic effect was abolished in the VP-LXRα transgenic mice (FIGS. 2h and 2i).

### EXAMPLE 3

This example demonstrates the molecular mechanism by which LXR can be shown to regulate EST.

The 4.2-kb (−4164 bp to +46 bp) 5′ regulatory sequences of the mEst gene were cloned by PCR using a template of mEst-containing bacterial artificial chromosome (BAC) clone (ID RP24-571N6) from the Children's Hospital Oakland Research Institute BACPAC Resource Center (Oakland, Calif.). Deletion mutants were generated by PCR-mediated mutagenesis. HepG2 cells were transfected with the reporter constructs and LXRα expression vector in 48-well plates as previously described . Gong, H. et al., Mol. Endocrinol. 20:279-290 (2006) When necessary, cells were treated with TO1317 (10 μM) for 24 h prior to luciferase assay. The transfection efficiency was normalized against the β-gal activities from a co-transfected CMX-β gal vector. The hydrodynamic liver transfection was performed as we previously described. Zhou, J. et al. J Biol Chem. 281: 15013-15020 (2006). EMSA were performed using in vitro transcribed and translated proteins as described previously. Saini, S. P. et al., Hepatology 41:497-505 (2005)

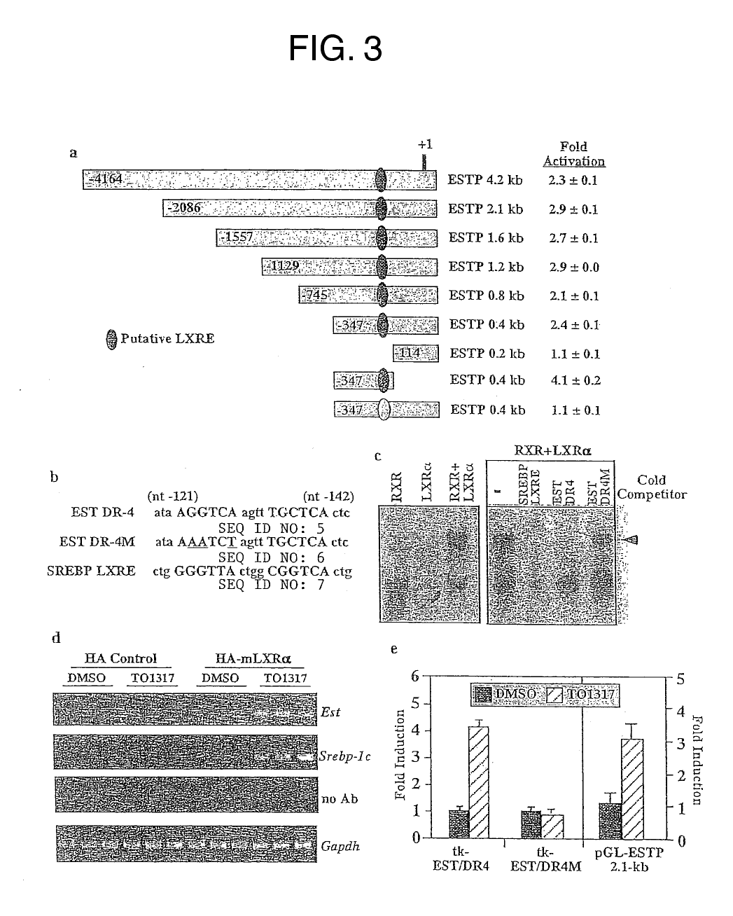

To examine the regulation of Est by LXRα, the 4.2-kb promoter sequences were activated by the wild type LXRα in the presence of TO1317 (FIG. 3a). Deletion analyses located the LXR responsive region to nt −200 bp to −400 bp (FIG. 3a). Inspection of this 200-bp sequence revealed a DR-4 (direct repeats spaced by four nucleotides) type NR response element (FIG. 3b). Electrophoretic mobility shift assays (EMSA) revealed that the LXRα/RXR heterodimers can bind to Est/DR-4, and this binding can be efficiently competed by excess unlabeled Srebp-1c/DR-4 (see Repa, J. J. et al., Genes Dev. 14:2819-2830 (2000)) or Est/DR-4 but not by the mutant Est/DR-4 (FIG. 3c).

Chromatin immunoprecipitation (CHIP) assay was used to demonstrate the recruitment of LXRα onto the Est promoter. In this experiment, the HA-tagged mouse LXRα or the HA vector control plasmid was transfected into the livers of the wild type mice in the presence or absence of TO1317 treatment. CHIP assay was performed with the use of an anti-HA antibody. Zhou, J. et al. J Biol Chem. 281: 15013-15020 (2006). Four-week old wild type female mice received an i.p. injection of DMSO or TO1317 (50mg/kg) 30 min before being hydrodynamically transfected with the pCMX-HA-LXRα or pCMX-HA control plasmid. The liver transfection and CHIP assays were performed the same as described previously. Zhou, J. et al. J Biol Chem. 281: 15013-15020 (2006). The primers for mEst/DR-4 are: 5′-CCAAAGGGGAGAAACAGCTG-3′ (SEQ ID NO:1) and 5′-GAGAAGGAGGCAGAGACTAAC-3′ (SEQ ID NO:2); Primers for mSrebp-1c/DR-4 are: 5′-CTCTTTTCGGGGATGGTTG-3′(SEQ ID NO:3) and 5′-GGTTTCTCCCGGTGCTCT-3′ (SEQ ID NO:4). The PCR products of Est and Srebp-1c are 142-bp and 141-bp, respectively.

As shown in FIG. 3d, treatment with TO1317 resulted in the recruitment of HA-LXRα onto the Est promoter. CHIP on the Srebp-1c gene promoter was included as the positive control. Consistent with EMSA results, tk-Est/DR-4, an Est/DR-4 containing synthetic thymidine kinase (tk) reporter, but not its mutant variant, was activated by LXRα in the presence of TO1317 (FIG. 3e). The DR-4 element is also necessary in the context of the 0.4-kb natural promoter, as mutation of this element abolished LXRα transactivation (FIG. 3a). The 2.1-kb natural promoter was also activated by TO1317 when transfected into the mouse liver (FIG. 3e).

### EXAMPLE 4

This example demonstrates that activation of LXR can inhibit estrogen-promoted breast cancer growth in xenograft models using ovariectomized nude mice.

Ovariectomized nude mice were purchased from Taconic (Germantown, N.Y.). ER-positive and estrogen responsive breast cancer MCF-7 cells were used. The MCF-7 cells were MCF-7/VEGF cells that overexpress the vascular endothelial growth factor (VEGF), rather than parent MCF-7 cells. MCF-7/VEGF cells have been found to exhibit a full penetrance of both estrogen independent and dependent growth with substantially increased tumor volumes in the presence of E₂. Guo, P. et al., Cancer Res. 63:4684-4691 (2003).

MCF-7 breast tumors were established in the mammary fat pads of ovariectomized female nude mice as described previously. Briefly, 1×10⁷ of MCF-7/VEGF cells were inoculated into the mammary fat pads of 8-week old ovariectomized female nude mice that were implanted with E₂ pellets (0.72 mg/60-day release) or placebo pellets (Innovative Research of America, Sarasota, Fla.). The E₂-treated mice were randomly divided into two groups, with one group receiving daily treatment of TO1317 (15 mg/kg by gavage) and the other receiving vehicle. The volumes of the tumors were measured using a caliper every five days. Mice were labeled with BrdU 30 min prior to sacrificing. The serum concentrations of E₂ were measured using the ACTIVE® ESTRADIOL EIA kit (Diagnostic Systems Laboratories, Webster, Tex.).

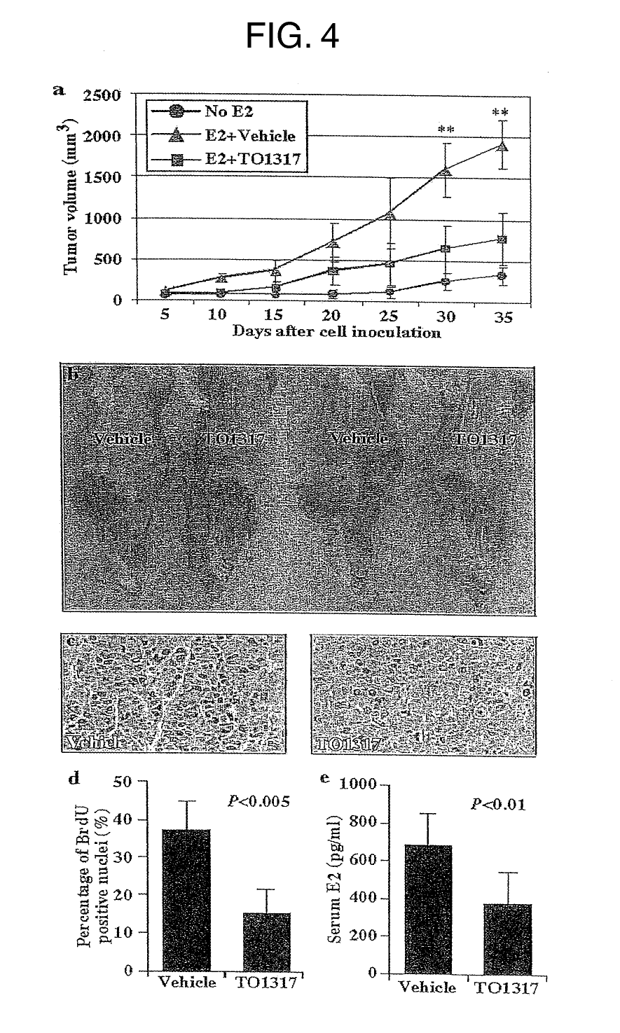

The estrogen effects on the tumorigenicity of the MCF-7/VEGF cells were evaluated and compared in the absence or presence of TO1317. As shown in FIG. 4, the MCF-7/VEGF cells were highly tumorigenic in E₂-treated mice, but TO1317 treatment significantly attenuated the E₂-enhanced tumor growth, leading to tumor growth kinetics similar to that in the absence of E₂ treatment (FIG. 4a). FIG. 4b shows the representative E2-induced tumors in absence or presence of TO1317. The tumor inhibitory effect was associated with a decreased tumor cell BrdU labeling index (FIGS. 4c and 4d) and circulating E₂ levels (FIG. 4e) in the TO1317-treated tumor bearing mice. The hepatic EST expression in the TO 1317-treated nude mice was also significantly induced compared to their mock-treated counterparts (data not shown).

### EXAMPLE 5

This example demonstrates that estrogen EST is a transcriptional target of GR, and that EST can be increased upon activation of GR by administration of GR agonists.

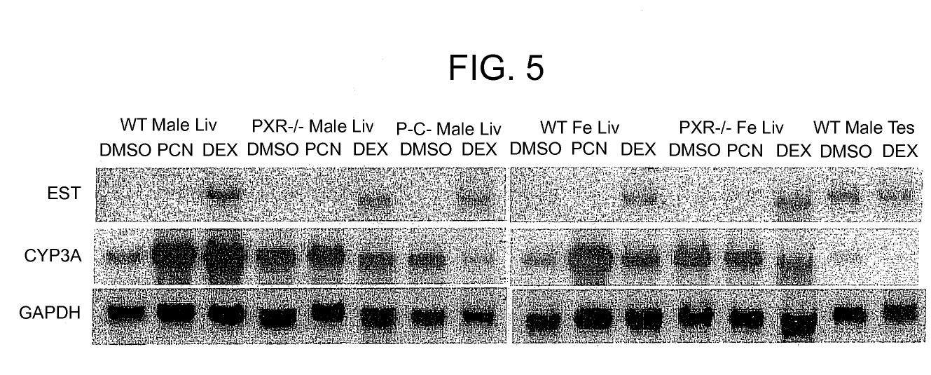

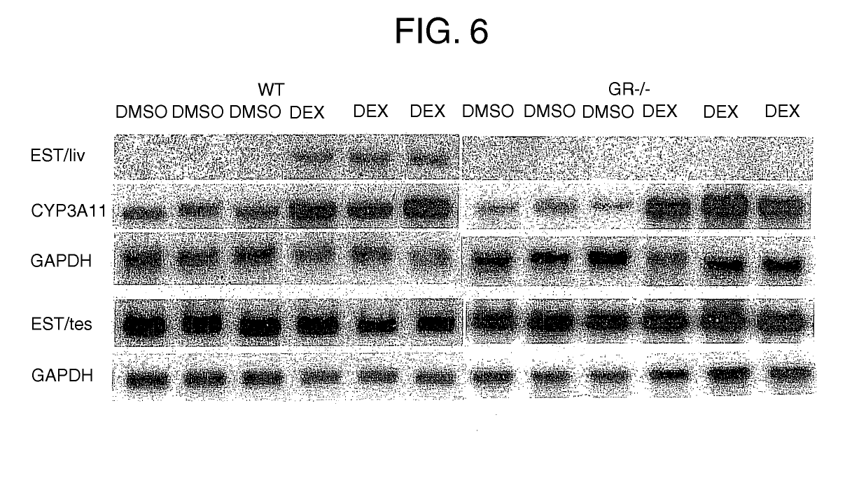

Following the protocols described in Example 1, administration of the GR agonist dexamethasone increased mRNA expression of EST compared to controls of DMSO and pregnenolone-16alpha-carbonitrile (PCN). The up-regulation of EST was observed in wild-type liver cells of mice of both sexes. (FIG. 5). EST was not up-regulated in GR knockout mice treated with dexamethasone. (FIG. 6)

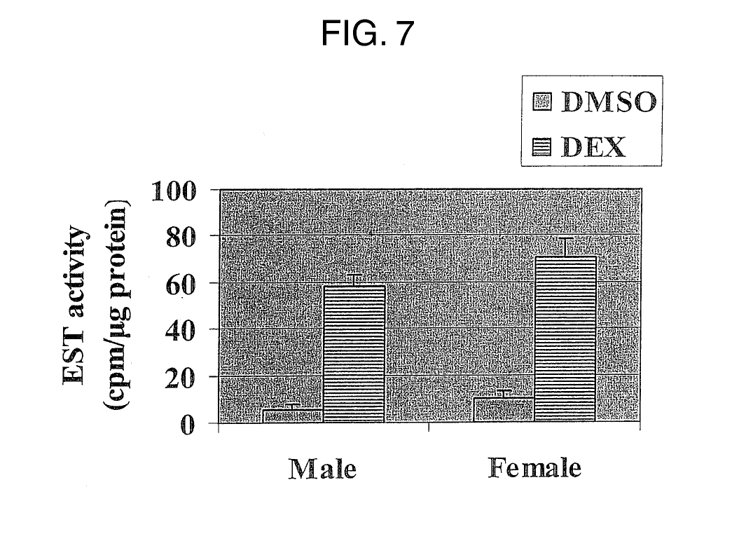

The effects of genetic and pharmacological activation of GR on EST expression were also confirmed by a sulfotransferase enzymatic assay using estrone as the substrate, as described in Example 1. (FIG. 7)

### EXAMPLE 6

This example demonstrates that activation of GR can inhibit estrogen-promoted breast cancer growth in xenograft models using ovariectomized nude mice.

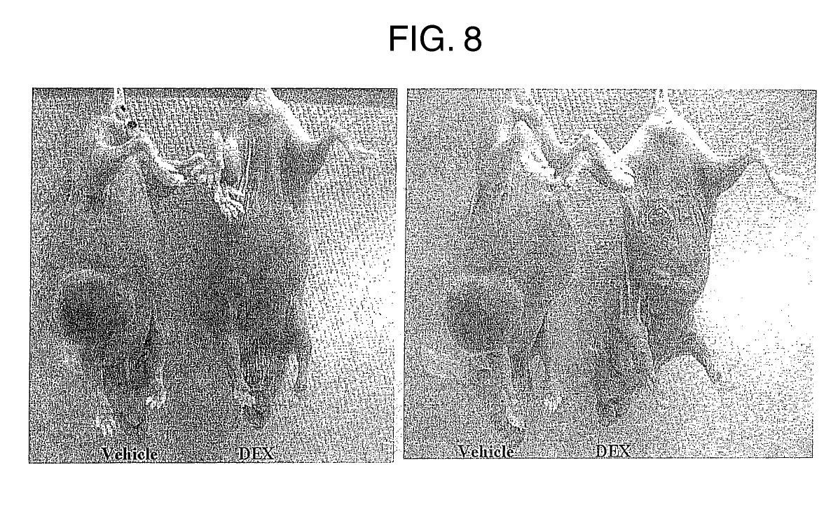

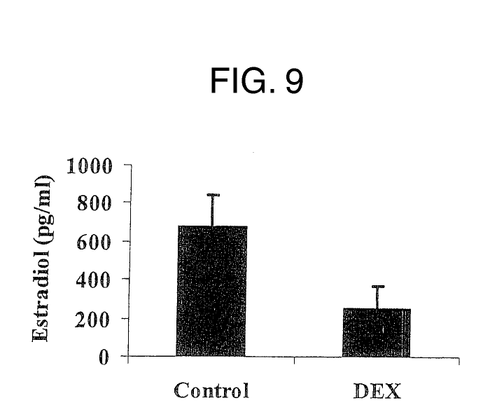

Breast tumors were established as described in Example 4, using MCF-7/VEGF cells. The estrogen effects on the tumorigenicity of the MCF-7/VEGF cells were evaluated and compared in the absence or presence of dexamethasone. FIG. 8 shows the representative E2-induced tumors in absence or presence of dexamethasone. The tumor inhibitory effect was associated with circulating E₂ levels (FIG. 9) in the dexamethasone-treated tumor bearing mice. The MCF-7/VEGF cells were highly tumorigenic in E₂-treated mice, but tumor growth and serum estradiol levels were significantly attenuated in the treated mice as compared to the controls.

### EXAMPLE 7

This example demonstrates that expression of EST in human cells can be increased upon administration of GR agonists.

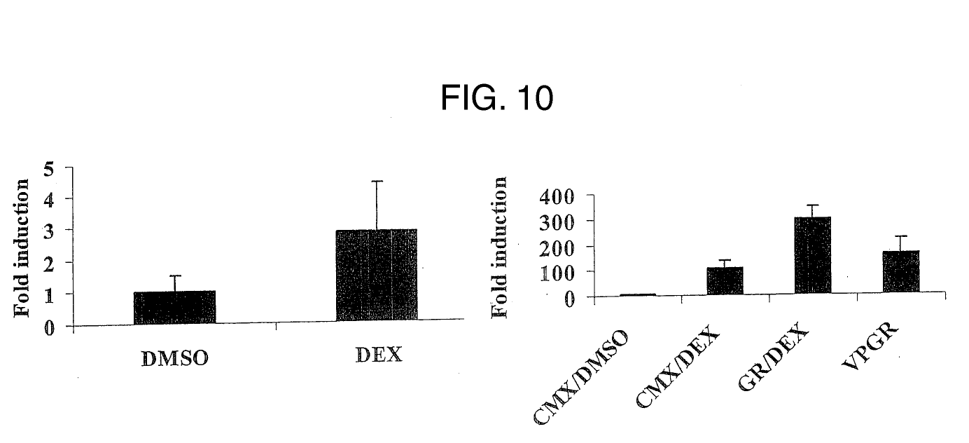

Expression of EST MRNA was measured as described above in human hepatocytes and in human breast cancer line MCF7. FIG. 10(a) shows the increased induction of EST in hepatocytes treated with dexamethasone as compared to a DMSO control. FIG. 10(b) shows increased induction of EST in MCF7 breast cancer cells treated with dexamethasone that have been transfected with empty vector CMX, GR, or the activated VPGR, treated with dexamethasone as compared with the solvent DMSO.

### EXAMPLE 8

This example demonstrates that combination treatment comprising an agonist of LR and an agonist of GR can increase the expression of EST over the levels provided by either agonist alone.

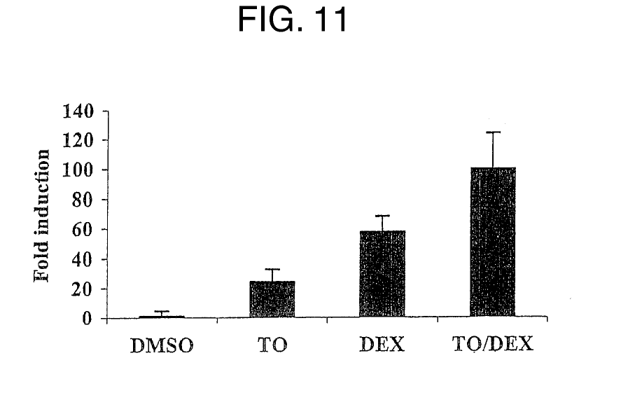

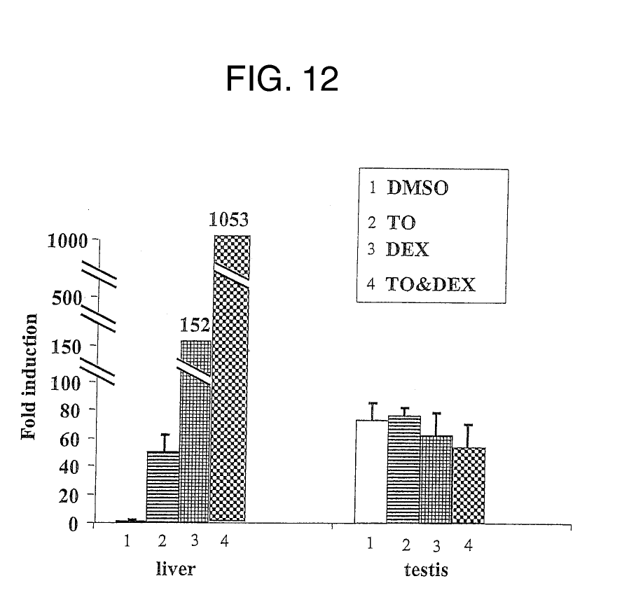

Expression of EST was measured as described above in male and female CD-1 mice treated with TO1317, dexamethasone, a combined injection of TO1317 and dexamethasone or a DMSO control. Expression of EST was greatly increased in livers of both male and female mice treated with the combination as compared to single-agonist treatment or DMSO control. (FIGS. 11 and 12) Up-regulation of EST was not observed in conjunction with combination treatment or single-agonist treatment in the testis of male mice. (FIG. 12).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of reducing the size of an estrogen responsive tumor comprising administering an agonist of an orphan nuclear receptor, wherein activation of the orphan nuclear receptor inhibits estrogen activity.

2. The method of claim 1, wherein the estrogen responsive tumor is associated with a condition selected from the group consisting of breast cancer, lung cancer, uterine cancer, and prostate cancer.

3. The method of claim 1, wherein the nuclear receptor is selected from the group consisting of Liver X Receptors (LXR) and glucocorticoid receptors (GR).

4. The method of claim 3, wherein the Liver X Receptor is selected from the group consisting of LXRα and LXRβ.

5. The method of claim 4, wherein the agonist is selected from the group consisting of GW3965, TO1317 (T0901317) and 22(R)-hydroxycholesterol.

6. The method of claim 3, wherein the nuclear receptor is GR, and wherein the agonist is a GR agonist.

7. The method of claim 6, wherein the agonist is selected from the group consisting of dexamethasone, ZK216348, RU28363, cortisol, prednisone, and prednisolone.

8. The method of claim 1, comprising administration of a first agonist of a first orphan nuclear receptor, and administration of a second agonist of a second orphan nuclear receptor, wherein the first agonist is different from the second agonist.

9. The method of claim 8, wherein the first orphan nuclear receptor is different from the second orphan nuclear receptor.

10. The method of claim 8, wherein the first orphan nuclear receptor is LXR and the second orphan nuclear receptor is GR.

11. The method of claim 10, wherein the first agonist is GW3965 and the second agonist is dexamethasone.

12. The method of claim 1, further comprising administration of chemotherapy.

13. The method of claim 12, wherein the chemotherapy is selected from the group consisting of an antiestrogen, an aromatase inhibitor, and a cytotoxic chemotherapy.

14. The method of claim 12, wherein the chemotherapy comprises an antiestrogen selected from the group consisting of tamoxifen, toremifene, raloxifene, droloxifene, idoxifine, nafoxidine, levomeloxifene, clomiphene, CI-680, CI-628, CN-55,956-27, MER-25, U-11,555A, U-11,100A, ICI-46,669, ICI-46,474, diphenolhydrochrysene, erythro-MEA, Parke Davis CN-35,945, allenolic acid, cyclofenil, ethamoxytriphetol, triparanol.

15. The method of claim 12, wherein the chemotherapy comprises an aromatase inhibitor selected from the group consisting of letrozole, anastrozole, exemestane, raloxifene, fadrozole, lentaron, formestane, rivizor, vorozole, and fulvestrant.

16. The method of claim 12, wherein the chemotherapy comprises a cytotoxic chemotherapy selected from the group consisting of methotrexate, cyclophosphamide, 5-flurouracil, doxorubicin, paclitaxel, and docetaxel.

17. The method of claim 1, wherein the agonist is administered as an adjuvant therapy to surgery.

18. A method of treating an estrogen responsive condition comprising administration of an agonist of an orphan nuclear receptor, wherein activation of the orphan nuclear receptor inhibits estrogen activity.

19. The method of claim 18, wherein the estrogen responsive condition is selected from the group consisting of breast cancer, lung cancer, uterine cancer, and prostate cancer.

20. A pharmaceutical composition comprising an agonist of one or more nuclear receptors and a pharmaceutically acceptable vehicle, wherein the nuclear receptor is an inhibitor of estrogen activity.

21. The composition of claim 20, wherein the nuclear receptor is selected from the group consisting of Liver X Receptors (LXR) and glucocorticoid receptors (GR).

22. The composition of claim 21, wherein the Liver X Receptor is selected from the group consisting of LXRα and LXRβ.


SEQUENCE LISTING (7 sequences)

SEQ ID NO: 1 - DNA, 20 bp, Artificial sequence
  Primer
ccaaaggggagaaacagctg

SEQ ID NO: 2 - DNA, 21 bp, Artificial sequence
  Primer
gagaaggaggcagagactaac

SEQ ID NO: 3 - DNA, 19 bp, Artificial sequence
  Primer
ctcttttcggggatggttg

SEQ ID NO: 4 - DNA, 18 bp, Artificial sequence
  Primer
ggtttctcccggtgctct

SEQ ID NO: 5 - DNA, 22 bp, Artificial sequence
  partial sequence of mEst gene promoter
ataaggtcaagtttgctcactc

SEQ ID NO: 6 - DNA, 22 bp, Artificial sequence
  partial sequence of mEst gene promoter
ataaaatctagtttgctcactc

SEQ ID NO: 7 - DNA, 22 bp, Artificial sequence
  LXRE from the Srebp-1c promoter
ctggggttactggcggtcactg